(19) United States
(12) Patent Application Publication
(10) Pub. No.: US 2015/0265278 A1
(43) Pub. Date: Sep. 24, 2015

(54) CLOSURE DEVICES, SYSTEMS, AND METHODS FOR TREATING BODY TISSUE

(71) Applicant: Boston Scientific Scimed, Inc., MAPLE GROVE, MN (US)

(72) Inventors: William BERTOLINO, Framingham, MA (US); Peter L. Dayton, Brookline, MA (US); Jennifer Marie Mehl, Watertown, MA (US)

(21) Appl. No.: 14/659,760

(22) Filed: Mar. 17, 2015

Related U.S. Application Data
(60) Provisional application No. 61/955,371, filed on Mar. 19, 2014.

(51) Int. Cl. A61B 17/08; A61B 17/00; A61B 17/064

(52) U.S. Cl. CPC A61B 17/085; A61B 17/0644; A61B 17/00234; A61B 17/0057; A61B 2017/081; A61B 2017/0647; A61B 2017/088; A61B 2017/00668; A61B 2017/00584

(57) ABSTRACT

A tissue closure device includes a first mesh, a second mesh, and a locking element. The first mesh is sized and shaped to be adhered to a first portion of tissue along a first edge of a tissue opening. The second mesh element is sized and shaped to be adhered to a second portion of tissue along a second edge of the tissue opening substantially opposing the first edge so that the drawing the first and second elements toward one another closes the tissue opening. The locking element is configured to draw the first and second mesh elements toward one another to a sealing position sealing the tissue opening and to lock the first and second mesh elements in the sealing position.

### PRIORITY CLAIM

The present application claims priority to U.S. Provisional Patent Application Ser. No. 61/955,371 field Mar. 19, 2014; the disclosure of which is incorporated herewith by reference.

### FIELD OF THE DISCLOSURE

The present disclosure relates to systems and methods for treating body tissue, more particularly to closure devices, systems, and methods for treating openings in body tissue.

### BACKGROUND

A wide variety of medical techniques are used for diagnosis as well as treatment within a patient's body, such as in the gastrointestinal tract. These techniques may involve severing and removing malignant or non-malignant tissue such as polyps. Generally, these techniques result in perforations in the tissue that may lead to bleeding, leakage, infections, patient discomfort, or other complications. Perforations may also result from endoscopic procedures (e.g., colonoscopies), closures in NOTES (Natural Orifice Transluminal Endoscopic Surgery) procedures, sealing after the removal of an organ or tissue unrelated to malignant or benign growths such as removal of the gall bladder (cholecystectomy), or attaching tissues so as to reshape (e.g., reshaping the fundus of the stomach as with GERD-fundoplication or hernia repair). In addition, a patient may require treatment for tissue openings associated with lesions or other defects within the body.

To avoid complications and promote healing, tissue openings may require closure. A variety of surgical clips such as hemostasis clips, and sutures are commercially available for closing tissue openings. However, large perforations may not be adequately treated with available hemostasis clips and suturing options. In addition, these conventional devices may not be suitable for endoscopic use, laporoscopic or minimally invasive “keyhole” or natural orifice procedures.

Therefore, there is an on-going need for devices and related methods that may be used for closure of tissue opening effectively and efficiently.

### SUMMARY OF THE DISCLOSURE

The present disclosure is directed to a tissue closure device including a first mesh, a second mesh, and a locking element. The first mesh element is sized and shaped to be adhered to a first portion of tissue along a first edge of a tissue opening. The second mesh element is sized and shaped to be adhered to a second portion of tissue along a second edge of the tissue opening substantially opposing the first edge thereby drawing the first and second elements toward one another closes the tissue opening. The locking element is configured to draw the first and second mesh elements toward one another to a sealing position sealing the tissue opening and to lock the first and second mesh elements in the sealing position.

In another embodiment, the present disclosure is directed to a method for treating a tissue opening. The method includes adhering a first mesh element to a first portion of tissue along a first edge of a tissue opening and then adhering a second mesh element to a second portion of tissue along a second edge of the tissue opening substantially opposing the first edge. The method further includes drawing the first and second mesh elements toward one another into a tissue-closing configuration and locking the first and second mesh elements in the tissue-closing configuration.

In another embodiment, the present disclosure is directed to a tissue closure system includes a grasping device and a first rivet. The grasping device includes a first arm and a second arm movable relative to one another between an open configuration and a closed configuration. In the open configuration, the first and second arms are separated from one another to receive opposing edges of a tissue opening therebetween. In the closed configuration, the first and second arms are drawn together to draw the opposing edges of the tissue opening toward one another. The first rivet is housed within the first arm at a distal end thereof. The first rivet includes a head portion and a shaft extending from the head portion such that the shaft extends through the first arm into a tissue-receiving space between the first and second arms. When the first and second arms are moved to the closed configuration, the shaft pierces through the opposing edges of the tissue opening. The shaft is movable to a locked configuration permitting a tip thereof to hold the opposing edges of the tissue opening together upon deployment of the first rivet from the grasping device.

It is to be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 shows a schematic top view of components of a tissue closure device.

FIG. 2 shows a schematic side view of the locking element of the tissue closure device of FIG. 1 in an open configuration.

FIG. 3 shows a schematic side view of the locking element of the tissue closure device of FIG. 1 in a closed configuration.

FIG. 4 shows a schematic side view of an exemplary embodiment of a tissue closure system in an open configuration.

FIG. 5 shows a schematic side view of an exemplary embodiment of a rivet of the tissue closure system of FIG. 4 before deployment.

FIG. 6 shows a schematic view of the rivet of FIG. 5 upon deployment.

### DETAILED DESCRIPTION

The present disclosure may be further understood with reference to the following description and the appended drawings, wherein like elements are referred to with the same reference numerals. Exemplary embodiments of the present disclosure describe systems and methods for treating tissue. In particular, exemplary embodiments describe devices and methods for treating openings or perforations in the tissue e.g., caused by intraluminal procedures such as Endoscopic Mucosal Resection (EMR), Polypectomy, Mucosectomy, or the like. The exemplary devices and methods may be used to seal or close openings or perforations in the body tissue without use of suture or electro-cautery.

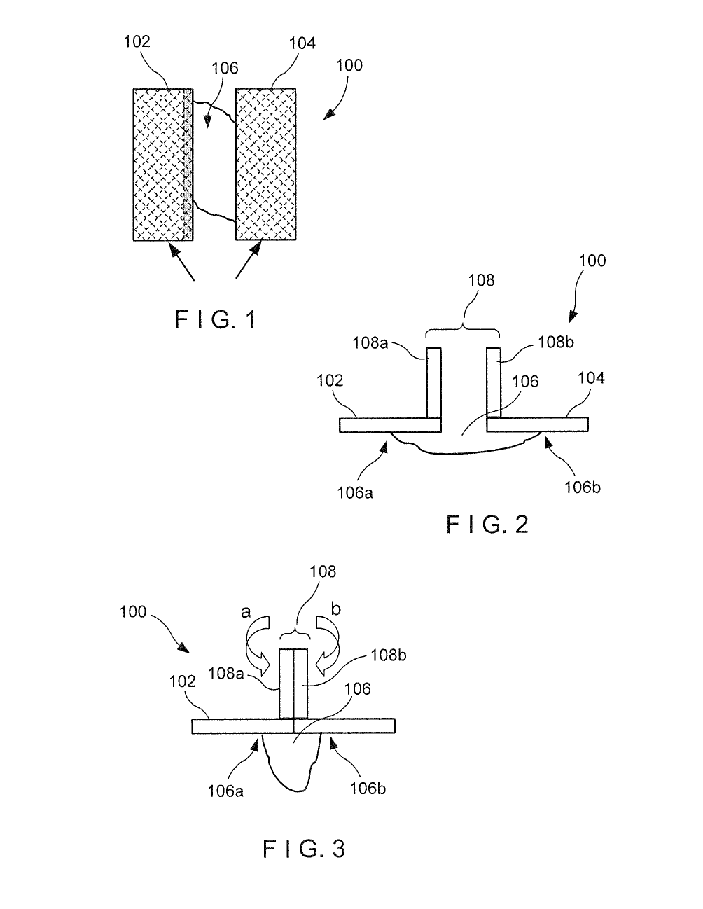

As shown in FIGS. 1-3, a tissue closure device 100 according to an exemplary embodiment of the present disclosure comprises a first mesh element 102 and a second mesh element 104 that can be used to close a tissue opening 106. The first element 102 is adhered to a first portion of tissue along a first edge (first edge 106a as shown in FIG. 2) of the tissue opening 106 and the second mesh element 104 is adhered to a second portion of tissue along a second edge (second edge 106b as shown in FIG. 2) of the tissue opening 106 substantially opposite the first edge. The first and second mesh elements 102, 104 are then drawn toward one another and locked relative to one another via a locking element 108 to close the tissue opening 106. Although the exemplary embodiment shows and describes the first and second elements 102, 104 as a mesh material, it will be understood by those of skill in the art that the first and second elements 102, 104 may be formed of any of a variety of materials so long as the first and second elements 102, 104 cover portions of tissue along edges of a tissue opening 106.

As shown in FIG. 1, the first and second mesh elements 102, 104, respectively, may be placed substantially parallel to the one another on opposite sides of a tissue opening so that a gap is defined between the first and second mesh elements 102, 104. It will be understood by those of skill in the art, however, that the first and second mesh elements 102, 104 may be placed over tissue in any of a variety of configurations so long as the first and second mesh elements 102, 104, respectively, may be drawn toward one another to close or narrow the gap between them, thereby closing the tissue opening 106. Thus, the tissue opening may be sealed by drawing together the edges of the tissue opening or by the drawing together the edges of the mesh element 102, 104 so that the tissue opening is entirely covered thereby. The mesh of the elements 102, 104 promote tissue ingrowth to further aid in sealing the tissue opening.

The first and second mesh elements 102, 104 are chosen to have dimensions such that the first and second mesh elements 102, 104 have surface areas sufficient to maintain a desired grip on the underlying tissue—i.e., sufficient to prevent their being inadvertently separated from the underlying tissue. In some embodiments, the shape and size of the first and second mesh elements 102, 104 may be selected based on factors such as, but not limited to, size of the tissue opening, location of the tissue opening, physical qualities of the surrounding tissue, or the like. The first and second mesh elements 102, 104 may have a same or different shape and size and may be modified by a user in any way suitable to a particular procedure so long as sufficient gripping is maintained between the mesh elements and the underlying tissue. A material of the first and second mesh elements 102, 104 may be selected to promote a tissue ingrowth and reduce infections.

The first and second mesh elements 102, 104 may be formed from any suitable biocompatible material such as but not limited to polymers, plastics, metals, alloys, or the like. In some embodiments, the first and second mesh elements 102, 104 are formed from plastics such as, but not limited to, Polyvinylchloride (PVC), Polyetheretherketone (PEEK), Polypropylene, Polyethylene, or copolymers of these, or the like. In other embodiments, the first and second mesh elements 102, 104 may be formed from a biodegradable material such as polylactic acid, polyglycolic acid, or the like. The first and second mesh elements 102, 104 may also be formed of biodegradable materials selected to degrade within the body after a time sufficient for healing has elapsed. As will be understood by those skilled in the art, removal of such mesh elements is not required.

The first and second mesh elements 102, 104 may include gaps and perforations allowing tissue growth therethrough to further secure the mesh elements 102, 104 to the tissue. In some embodiments, the first and second mesh elements 102, 104 may be coated with an active pharmaceutical agent for promoting tissue growth or fighting infection.

An adhesive or sealing material may be applied to the first and second mesh elements 102, 104 to adhere these elements to the underlying tissue. For example, any suitable medical-grade adhesive may be applied over the first and second mesh elements 102, 104 including, but not limited to, epoxy resins, acrylic resins, polyurethane adhesives, colloidal epoxy silica, or the like. In addition, the surfaces of the first and second mesh elements 102, 104 may have features, such as micro-abrasions, increasing the gripping between these elements and the underlying tissue.

As shown in FIGS. 2-3, a locking element 108 may be coupled to the first and second mesh elements 102, 104 to draw these elements toward one another and, when a desired position of these elements relative to one another has been achieved, to lock the first and second mesh elements 102, 104 in this desired position (e.g., to seal a tissue opening 106). The locking element 108 may be, for example, a clip, such as a hemostasis clip, including a first arm 108a and a second arm 108b. A distal end of the first arm 108a grips a portion of the first mesh element 102 while a distal end of the second arm 108b grips a second portion of the second mesh element 104 so that as the first and second arms 108a, 108b are drawn toward one another, the first and second mesh elements 102, 104 are also drawn toward one another to close the gap therebetween.

The first and second arms 108a, 108b, respectively, of the locking element 108 are movable between an open configuration, shown in FIG. 2, and a closed configuration, shown in FIG. 3. In the open configuration, the first arm 108a and the second arm 108b are separated from one another such that the distal ends of the arms 108a, 108b may be positioned over corresponding portions of the first and second mesh elements 102, 104, respectively. Once the distal ends of the arms 108a, 108b have grasped corresponding portions of the first and second mesh elements 102, 104, the locking element 108 is moved to the closed configuration, in which the first arm 108a and the second arm 108b are drawn toward one another. This draws the first and second mesh elements 102, 104 toward one another drawing together the underlying tissue to which they are coupled to close the tissue opening 106. An operator may move the first and second arms 108a, 108b of the locking element 108 towards one another in directions a and b (as shown in FIG. 3) to close the tissue opening 106. Once sealed, the first and second arm 108a, 108b may be locked in the closed configuration to ensure complete closure of the tissue opening 106. It will be understood by those of skill in the art that a clip locking element 108 may include any of a variety of mechanisms for holding the arms 108a, 108b together in the locked configuration. For example, the clip may include a spring or other means biasing the locking element 108 toward the closed configuration.

The locking element 108 may be designed to allow for ease in drawing the mesh elements 102, 104 together to close the gap between the arms 108a and 108b. For that purpose, the first and second arms 108a, 108b may be made stiff and of sufficient size to allow for easy actuation.

Although the exemplary embodiments show and describe the locking element 108 including arms 108a and 108b, it will be understood by those of skill in the art that the locking element 108 may include any of a variety of locking mechanisms so long as the locking element 108 is capable of drawing the first and second mesh elements 102, 104 together and locking them in this position to maintain the tissue opening 106 in a closed or sealed configuration. For example, in another embodiment, the locking element 108 may be a zip fastener. The zip fastener may include a first interlocking member coupled to a portion of the first mesh element 102 and a corresponding second interlocking member coupled to a portion of the second mesh element 104. The first and second interlocking members may include coupling elements that, when drawn together upon actuation of the locking element 108, couple with each other to secure the first and second mesh elements 102, 104 to each other in a sealing position.

In yet another embodiments, the locking element 108 may include a hook and loop fastener. The hook may be coupled to the first mesh element 102 and the loop fastener may be coupled to the second mesh element104. The first and second mesh elements 102, 104 may be drawn closer towards one another to close the tissue opening 106 by drawing the hook and the loop fastener and may be kept engaged by securing the hook to the loop fastener. As would be understood by those skilled in the art, the hook and the loop fastener may include complementary mating members with complementary mating surfaces including male and female counters to remain engaged with each other.

In another embodiments, the locking element 108 may include an adhesive tab extending along a portion of at least one of the first and second mesh elements 102, 104. The adhesive tab may be designed to seal and lock the first and second mesh elements 102, 104. In some embodiments, the adhesive tab may extend along a portion of either the first mesh element 102 or the second mesh element 104 so that, when placed in contact with the other mesh element, the adhesive tab is adhered thereto locking the first and second mesh elements 102, 104 in the sealing position sealing the tissue opening 106. In some embodiments, one adhesive tab may be coupled to each of the first mesh element 102 and the second mesh element 104 to further ensure a good seal.

According to an exemplary method for treating a tissue opening 106 using the device 100, a first mesh element 102 is adhered to a first portion of tissue along a first edge 106a of the tissue opening 106. Then, a second mesh element 104 is adhered along a second edge 106b of the tissue opening 106 such that the second mesh element 104 is disposed substantially opposing the first edge. Where the locking element 108 is a clip, the locking element 108 may be positioned over the tissue opening 106 in the open configuration such that such that a distal end of the first arm 108a grasps a portion of the first mesh 102 while the distal end of the second arm 108 grasps a portion of the second mesh element 104. The locking element 108 is then moved to the closed configuration to draw the first and second mesh elements 102, 104 toward one another to a tissue-closing configuration. In the tissue-closing configuration, the first and second edges 106a, 106b of the tissue opening 106 are drawn toward one another to close the tissue opening 106. The locking element 108 is then locked, holding the tissue opening 106 in the tissue-closing configuration. The first and second mesh elements 102, 104 may be locked temporarily or permanently, and the duration of locking may depend upon time taken for the tissue opening 106 to close permanently. Although the exemplary method describes the locking element 108 as a clip, it will be understood by those of skill in the art that the first and second mesh elements 102, 104 may also be locked relative to one another in the tissue-closing configuration via a variety of other locking mechanisms such as, for example, a zip fastening mechanism including first and second interlocking members, an adhesive tab extending over a portion of at least one of the first and second mesh elements 102, 104, and a hook and loop fastener. In another embodiment, one or both of the first and second mesh elements 102, 104 may include a flexible member (e.g., string) mating with a slit formed through an opposing one of the first and second mesh elements 102, 104.

In some embodiments, the tissue closure device 100 may be used to close a tissue opening 106 within a body lumen such as esophagus, intestine, or the like. In such scenarios, the tissue closure device 100 may be used in conjunction with an elongated sheath e.g., an endoscope to access the tissue opening 106. The elongated sheath may include a lumen extending from a proximal end to a distal end of the elongated sheath. Each of the first and second mesh elements 102, 104 may be rolled up and inserted into the lumen of the sheath or endoscope in series so that the first and second mesh elements 102, 104 are deployed at the target area within the body one at a time via, for example, a pusher rod. Further, an operating member such as a push-pull wire, retractor, or the like may be deployed to control the tissue closure device 100. Each of the first and second mesh elements 102, 104 may be rolled up and inserted into the lumen of the sheath or endoscope in series so that the first and second mesh elements 102, 104 are deployed at the target area within the body one at a time via, for example, a pusher rod. The operating member may enable the operator to transition the locking element 108 between the open configuration and the closed configuration using, for example, pull wires as would be understood by those skilled in the art.

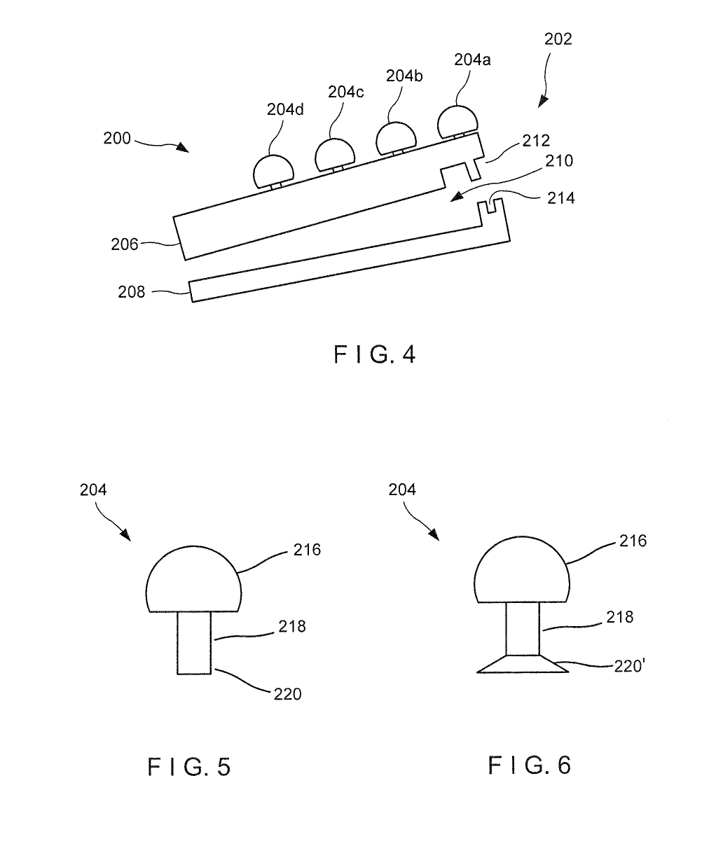

As shown in FIGS. 4-6, a tissue closure system 200 according to another exemplary embodiment of the present disclosure comprises a grasping device 202 and one or more rivets 204 (e.g., 204a, 204b, 204c, and 204d) housed within the grasping device 202. The tissue closure system 200 is configured to close a perforation or a tissue opening by partially or completely sealing opposing edges of the tissue opening together.

The grasping device 202 includes a first arm 206 and a second arm 208 such that the first arm 206 and the second arm 208 are movable relative to each other between an open configuration and a closed configuration. In the open configuration, the first arm 206 and the second arm 208 are separated from one another creating a tissue receiving space 210 therebetween. When in the open configuration, the tissue receiving space 210 is configured to receive opposing edges of the tissue opening. In contrast, the closed configuration is achieved when the two arms 206, 208 are drawn toward or against one another. In the closed configuration, the opposing edges of the tissue opening are drawn toward one another to seal or close the tissue opening. Instruments such as forceps may be used in conjunction with the grasping device to grasp opposing edges of the tissue opening and draw the opposing edges of the tissue opening between the two arms 206, 208 and into the tissue receiving space 210.

To accomplish closure of the tissue opening, the one or more rivets 204a, 204b, 204c, 204d may be deployed such that the opposing edges of the tissue opening may be pierced by the rivets 204a, 204b, 204c, 204d, thereby securing the opposing edges of the tissue together.

The rivet (e.g., one or more rivets 204a, 204b, 204c, 204d) includes a head portion (e.g., head portion 216 shown in FIG. 5) and a shaft (e.g., shaft 218 shown in FIG. 6) extending from the head portion. For example, the head portion 216 may be a partially circular portion disposed over a substantially cylindrical shaft 218. In another embodiment, the head portion 216 may be pre-formed in the shape of wings that are parallel to and against one another initially but which spring out perpendicular to the axis of the shaft when released from, for example, a tube. The shaft includes a tip (e.g., tip 220 as shown in FIG. 5) at its distal end that may be substantially pointed for easy piercing of the tissue. In some embodiments, the tip of the shaft may be tapered enabling easy insertion through the tissue without application of large amount of force.

As shown, the first arm 206 includes an opening 212 at its distal and the second arm 208 includes a complementary member 214 at its distal end. The opening 212 is sized and shaped to permit a shaft 218 of a first rivet 204a to extend through the first arm 206 to the tissue receiving space 210. When the first and second arms 206, 208 are drawn toward one another, the shaft of the first rivet 204a comes in contact with the complementary member 214 piercing the tissue (e.g., opposing edges of the tissue opening) in the tissue-receiving space 210. Then the complementary member 214 enlarges the tip of the first rivet 204a to a locked configuration thereby deploying the first rivet 204a and securing the opposing edges of the tissue opening.

To achieve this, in some embodiments, the complementary member 214 may include an electrode configured to heat the tip of the shaft thereby deploying the first rivet 204a to the locked configuration. The electrode may be a RF electrode that may heat the tip thereby enlarging the tip of the shaft (e.g., tip 220′ as shown in FIG. 6). In another embodiment, the complementary member 214 may include a locking ring that can be mounted over the shaft thereby deploying the first rivet 204a to the locked configuration. In yet another embodiment, the shaft may be formed from a shape memory alloy such that upon deployment, the first rivet 204a gets deployed into the locked configuration. In yet another embodiment, the shape of the first rivet 204a may be modified to the locked configuration by deformation of the tip by the application of sufficient force on the first and second arms 206, 208. In yet another embodiment, the complementary member 214 may include a cutting element. When the two arms 206, 208 are drawn close toward one another, the shaft of the first rivet 204a may come in contact with cutting element. The cutting element may cut a portion of the shaft extending to deploy the first rivet 204a in the locking configuration.

The first rivet 204a may be deployed in the body via a slot (not shown) extending from the opening 212 to an exterior of the distal end of the first arm 206 such that the opening 212 is open to an exterior of the grasping device 202. Thus, once the first rivet 204a is in the locking configuration, the first rivet 204a is moved distally with respect to the first arm 206, passing the rivet 204a through the slot to be deployed in the body.

Further, the first arm 206 of the grasping device 202 may include a ratchet system allowing sequential deployment of the multiple rivets 204a, 204b, 204c, 204d piercing the tissue. The ratchet system may be designed to resist jamming as well as unintentional or accidental deployment of the one or more rivets 204a, 204b, 204c, 204d. The ratchet system includes a pusher that pushes the first rivet 204a distally relative to the first arm 206. Once the first rivet 204a has been deployed, the grasping device is withdrawn and the pusher pushes another rivet (e.g., a second rivet 204b) distally to be deployed adjacent the first rivet 204a. For example, the second rivet 204b may be housed within the first arm 206 proximally of the first rivet 204a and movable to the distal end of the first arm 202 upon deployment of the first rivet 204a. Similar to the first rivet 204a, the second rivet 204b includes a head portion and a shaft extending therefrom so that, when the second rivet 204b is at the distal end of the first arm 206, the shaft of the second rivet 204b extends into the tissue-receiving space 210.

FIG. 5 shows an enlarged view of a rivet 204 before deployment in an unlocked configuration. FIG. 6 shows the rivet 204 upon deployment in a locked configuration. As shown, the tip 220′ of the shaft is enlarged. By enlarging the tip 220′ of the shaft 218, the rivet 204 is deployed in locked configuration such that once deployed the rivet 204 cannot be removed or drawn back.

In some embodiments, the head portion 216 and the shaft 218 may be integrally formed as a unitary structure. In other embodiments, the head portion 216 and the shaft 218 may be separately formed and then coupled together forming the rivet 204 using any suitable mechanical or chemical process. The rivet 204 may be formed using a suitable biocompatible material such as, but not limited to, stainless steel, aluminum, titanium, platinum, gold, silver, chromium, nickel, polyvinylchloride, polyurethane, Po lyetheretherketone, high density polyethylene, polyetherimide, polycaprolactone or their combination. In some embodiments, the rivet 204 may be formed from a biodegradable material that may get decomposed during the healing process.

It will be apparent to those skilled in the art that various modifications and variations may be made in the structure and the methodology of the present disclosure, without departing from the spirit or scope of the disclosure. Thus, it is intended that the present disclosure cover modifications and variations of the disclosure provided that they come within the scope of the appended claims and their equivalents.

What is claimed is:

1. A tissue closure device, comprising:
a first element sized and shaped to be adhered to a first portion of tissue along a first edge of a tissue opening;
a second mesh element sized and shaped to be adhered to a second portion of tissue along a second edge of the tissue opening substantially opposing the first edge so that drawing the first and second mesh elements toward one another closes the tissue opening; and
a locking element configured to draw the first and second mesh elements toward one another to a sealing position sealing the tissue opening and to lock the first and second mesh elements in the sealing position.

2. The device of claim 1, wherein the locking element is a clip including a first arm configured to grip the first mesh element and a second arm configured to grip the second mesh element, the first arm and the second arms being movable between an open configuration in which the first and second arms are separated from one another and a closed configuration in which the first and second arms are drawn toward one another to close the tissue opening.

3. The device of claim 1, wherein the locking element comprises a zip fastener including a first interlocking member along a portion of the first mesh element and a second corresponding interlocking member along a portion of the second mesh element such that actuation of the locking element draws the first and second mesh elements toward one another to the sealing position.

4. The device of claim 1, wherein the locking element comprises an adhesive tab extending along a portion of at least one of the first and second mesh elements.

5. The device of claim 1, wherein the locking element is a hook and loop fastener including a hook member extending along a portion of the first mesh element and a loop member extending along a portion of the second mesh element.

6. The device of claim 1, wherein the first and second mesh elements are treated with a medical-grade adhesive for adhering the first and second mesh elements to tissue.

7. The device of claim 1, wherein the first and second mesh elements are coated with a treatment for promoting tissue growth.

8. The device of claim 1, wherein the first and second mesh elements are comprised of a biodegradable material.

9. A method for treating a tissue opening, comprising:
adhering a first mesh element to a first portion of tissue along a first edge of a tissue opening;
adhering a second mesh element to a second portion of tissue along a second edge of the tissue opening substantially opposing the first edge; and
drawing the first and second mesh elements toward one another into a tissues-closing configuration and locking the first and second mesh elements in the tissue-closing configuration.

10. A tissue closure system, comprising:
a grasping device including a first arm and a second arm movable relative to one another between an open configuration in which the first and second arms are separated from one another to receive opposing edges of a tissue opening therebetween and a closed configuration in which the first and second arms are drawn together to draw the opposing edges of the tissue opening toward one another; and
a first rivet housed within the first arm at a distal end thereof, the first rivet including a head portion and a shaft extending therefrom such that the shaft extends through the first arm into a tissue-receiving space between the first and second arms so that, when the first and second arms are moved to the closed configuration, the shaft pierces through the opposing edges of the tissue opening, the shaft being movable to a locked configuration in which a tip thereof holds the opposing edges of the tissue opening together upon deployment of the first rivet from the grasping device.

11. The system of claim 10, wherein a tip of the shaft of the first rivet is tapered.

12. The system of claim 10, wherein the first arm includes a slot at a distal end thereof, the slot being sized and shaped to permit the first rivet to be passed therethrough when being deployed.

13. The system of claim 10, wherein the second arm includes an electrode at a distal end thereof which, when the grasping device is moved to the closed configuration and the shaft pierces the opposing edges of the tissue opening, heats the shaft tip to enlarge the tip and move the first rivet to the locked configuration.

14. The system of claim 10, wherein the second arm releasably houses a locking ring at a distal end thereof such that, when the grasping device is moved to the closed configuration and the shaft pierces the opposing edges of the tissue opening, the locking ring is mounted over the tip of the shaft to move the first rivet to the locked configuration.

15. The system of claim 10, wherein at least a portion of the shaft is formed of one of a shape memory material and a material having a predisposed shape such that the first rivet reverts to the locked configuration upon deployment.

16. The system of claim 10, wherein, in the locked configuration, the tip of the shaft is enlarged.

17. The system of claim 10, wherein the second arm includes a cutting element at a distal end thereof such that, when the grasping device is moved to the closed configuration and the shaft pierces the opposing edges of the tissue opening, the cutting element cuts a portion of the shaft extending from a far side of the tissue opening to permit the shaft to revert to the locked configuration.

18. The system of claim 10, wherein the first rivet is deployed via a pusher pushing the first rivet distally relative to the first arm.

19. The system of claim 10, wherein the first rivet is formed of a biodegradable material.

20. The system of claim 10, further comprising a second rivet housed within the first arm proximally of the first rivet and movable to the distal end of the first arm upon deployment of the first rivet, the second rivet including a head portion and a shaft extending therefrom so that, when the second rivet is at the distal end of the first arm, the shaft of the second rivet extends into the tissue-receiving space.
